(19) United States
(12) Patent Application Publication
(10) Pub. No.: US 2011/0158864 A1
(43) Pub. Date: Jun. 30, 2011

(54) Physiological Test Device

(75) Inventors: Jui-Lin Hsu, Taipei County (TW); Meng-Yi Lin, Taipei County (TW)

(73) Assignee: HEALTH & LIFE CO., LTD., Taipei County (TW)

(21) Appl. No.: 12/648,538

(22) Filed: Dec. 29, 2009

(51) Int. Cl. G01N 33/48

(52) U.S. Cl. 422/400

(57) ABSTRACT

The present invention discloses a physiological test device, which is used to test multiple physiological parameters in vitro and able to indicate the current test item with lights, sounds, images, text or a combination thereof lest the user be confused by the multiple functions of the test device or different test results thereof. The present invention can prevent the user from mistaking the test type or test result, and thus can overcome the conventional problems that the multiple functions of a single test device confuse the user. Thus, the user will not take a wrong test piece or obtain a wrong test result.

### BACKGROUND OF THE INVENTION

1. Field of the Invention

The present invention relates to a test device, particularly to a physiological test device, which tests multiple physiological parameters in vitro, and which uses lights and/or sounds to indicate the type of the test undertaken currently, whereby is solved the problem of recognizing multiple functions in a single test device.

2. Description of the Related Art

The current in-vitro physiological test device is gradually evolved into a composite test device, which integrates several physiological test functions and uses different test pieces to undertake different physiological tests.

The multiple functions in a single device may confuse the user. For example, the user may take a wrong test piece and thus have a wrong test result.

A Taiwan patent No. I253502 disclosed a “Nanometric Transistor Architecture of Biological Test Device and Manufacture Method Thereof”, which relates to a method for fabricating nanometric complementary transistors.

A Taiwan patent No. M291525 disclosed a “Disposable Biological Test Device”, wherein the liquid sample is dropped into the enzyme reaction region to enable the test.

A Taiwan patent No. 1279222 disclosed a “Multifunctional Portable Medical Test Device and Display Method Thereof”, which comprises a first test unit and a second test unit respectively undertaking different medical tests and generating a first test result and a second test result. After the tests, the user operates the button unit to output the first or second test result. The test unit, such as the glucometer, tonometer, cholesterol meter or uric acid meter, occupies a considerable volume. Further, the operation procedures are very complicated. Therefore, such a test device is unsuitable for ordinary users.

A Taiwan patent No. 00394776 disclosed a “DNA Sequence of Novel EB Virus Nuclear Antigen-1 Protein Segment, Applications Thereof, and Novel Fabrication Method Thereof”. The applications thereof include a test reagent containing the novel segment. The fabrication method includes a method to purify the protein or the segment thereof from the vectors or the host cells. Such a technology has little to do with a common physiological test device.

Therefore is eagerly desired a physiological test device, which tests multiple physiological parameters and indicates the type of the test undertaken currently, whereby the user will not be confused by the multiple functions of a single test device, and whereby the user will not take a wrong test piece or have a wrong test result, and whereby is solved the problem of recognizing the multiple functions in a single test device.

### SUMMARY OF THE INVENTION

The primary objective of the present invention is to provide a physiological test device, which tests multiple physiological parameters in vitro, and which uses lights, sounds, images, text or a combination thereof to indicate the type of the test undertaken currently.

Another objective of the present invention is to provide a physiological test device, which tests multiple physiological parameters in vitro, and which enables the user to recognize multiple test functions in a single test device.

A further objective of the present invention is to provide a physiological test device, which tests multiple physiological parameters in vitro, and which prevents the user from being confused by the multiple functions of a single test device, and which exempts the user from taking a wrong test piece and having a wrong test result.

According to the abovementioned objectives, the present invention proposes a physiological test device, which tests multiple physiological parameters in vitro, and which uses lights, sounds, images, text or a combination thereof to indicate the type of the test undertaken currently.

The physiological test device of the present invention comprises an insertion slot, a signal sensing unit, an identification unit, a control unit, an indicating unit and a display unit. The insertion slot receives a test piece or a memory card, and separately or simultaneously connects with the signal sensing unit and the identification unit. The signal sensing unit detects the signals generated by a test piece or a memory card in a test. The identification unit contains the information about all types of test pieces and memory cards, which are to be inserted into the insertion slot. The identification unit performs identification via recognizing the identification label on the test piece or reading the identification data in the memory card. The control unit receives and processes a sensation signal from the signal sensing unit to obtain a test result. The control unit also receives an identification result from the identification unit and instructs the indicating unit to present the corresponding indications according to the identification result. The indicating unit is controlled by the control unit to issue an indication signal to indicate the user of the type or result of a test that the physiological test device is performing on a test piece or a memory card. The indication signal may be lights, sounds, images or a combination thereof to highlight or present the type or result of the current test.

The indicating unit of the present invention may be a device, module, unit or element to statically or dynamically present lights, sounds, images, text, or a combination thereof.

The indicating unit of the present invention uses sounds, images or multimedia to statically or dynamically present the test results of different test pieces or memory cards.

Below, the embodiments are described in detail in cooperation with the attached drawings to make easily understood the objectives, characteristics and efficacies of the present invention.

### BRIEF DESCRIPTION OF THE DRAWINGS

FIG. 1 is a diagram schematically showing the architecture of a physiological test device according to the present invention;

FIG. 2 is a diagram schematically showing the image presented by a display unit of a physiological test device according to the present;

FIG. 3 is a diagram schematically showing the light presented by an indicating unit of a physiological test device according to the present invention; and

FIG. 4 is a diagram schematically showing that an indicating unit of a physiological test device issues vocal messages according to the present invention.

### DETAILED DESCRIPTION OF THE INVENTION

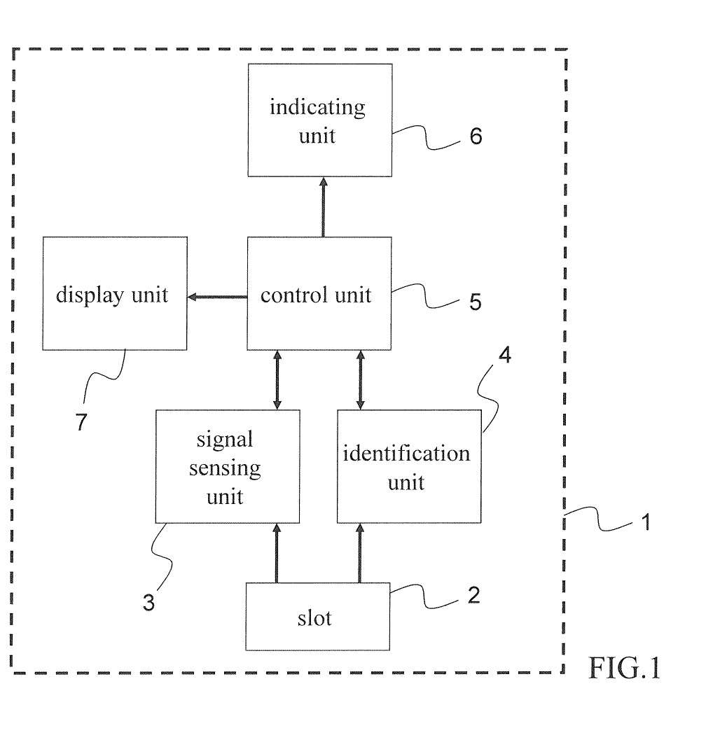

Refer to FIG. 1 a diagram schematically showing the architecture of a physiological test device according to the present invention. The physiological test device 1 of the present invention comprises an insertion slot 2, a signal sensing unit 3, an identification unit 4, a control unit 5, an indicating unit 6 and a display unit 7.

The insertion slot 2 receives a test piece or a memory card. The insertion slot 2 separately or simultaneously connects with the signal sensing unit 3 and the identification unit 4.

The signal sensing unit 3 electrically connects with the insertion slot 2, detects a signal generated by a test piece or a memory card in a test, and transfers the detection result to the control unit 5.

The identification unit 4 electrically connects with the insertion slot 2, identifying the type of the test piece or memory card inserted into the insertion slot 2. The identification unit 4 contains the information about all types of test pieces and memory cards, which are to be inserted into the insertion slot 2. The identification unit 4 performs identification via recognizing the identification label on the test piece or reading the identification data in the memory card. The identification may be realized via the logical signal generated by the identification label on the test piece. The logical signal generated by the identification label may be an electric signal, a mechanical signal or an optical signal. Besides, the identification information is not necessarily stored in the identification unit 4 itself but may be stored in a calibration card. The identification unit 4 transfers an identification result to the control unit 5.

The control unit 5 receives and processes the identification result and then performs the corresponding measurement and computation. The control unit 5 instructs the indicating unit 6 to present the corresponding indication according to the identification result. The control unit 5 receives and processes a sensation signal from the signal sensing unit 3 to obtain a test result.

The indicating unit 6 is controlled by the control unit 5 to indicate the type of a test that the physiological test device 1 is performing on a test piece or a memory card. The control unit 5 controls the indicating unit 6 to issue an indication signal and indicate the user of the type or result of the current test. The indication signal may be lights, sounds, images or a combination thereof to highlight or present the type or result of the current test. The indicating unit 6 can indicate the user of the type of the current test. The indicating unit 6 can also indicate the user of the type of the recorded data when the recorded data is called out.

The indicating unit 6 is a device, module, unit or element to statically or dynamically present lights, sounds, images, text, or a combination thereof. For examples, the indicating unit 6 uses different colors of lights to indicate different types of test pieces or memory cards; the indicating unit 6 uses lights at different positions of the appearance of the physiological test device 1 to indicate different test results of test pieces or memory cards; the indicating unit 6 uses different flashing frequencies of lights in the appearance of the physiological test device 1 to indicate different test results of test pieces or memory cards; the indicating unit 6 uses different music or audio effects to indicate different test results of test pieces or memory cards; the indicating unit 6 uses different vocal messages to indicate different test results of test pieces or memory cards.

The display unit 7 is an LCD (Liquid Crystal Display) panel or an OLED (Organic Light Emitting Diode) panel. The display unit 7 uses images to visually present the test results statically or dynamically. The images may be a part of a multimedia representation. In other words, the physiological test device can present the test results with multimedia.

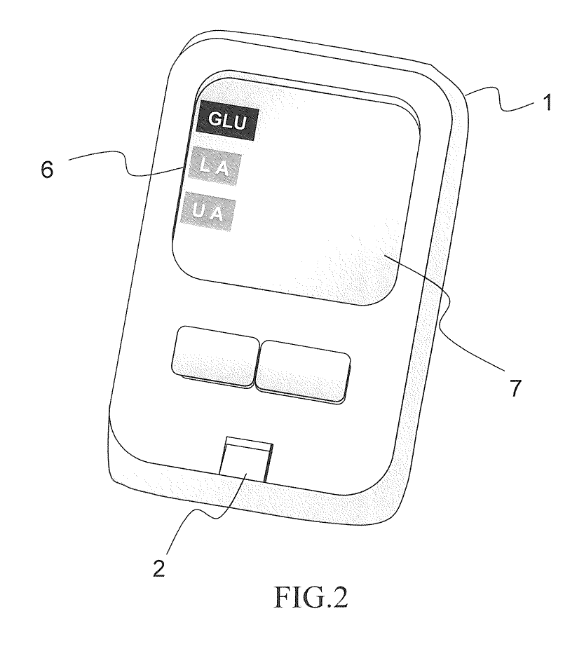

Refer to FIG. 2 a diagram schematically showing the image presented by a display unit of a physiological test device according to the present invention. As shown in FIG. 2, the physiological test device 1 has a display unit 7, and the display unit 7 has an indicating unit 6. The indicating unit 6 may be a device, module, unit or element to present/highlight images or text. The display unit 7 is an LCD or OLED panel to statically or dynamically display multimedia information to present the test types and test results of the physiological test device 1.

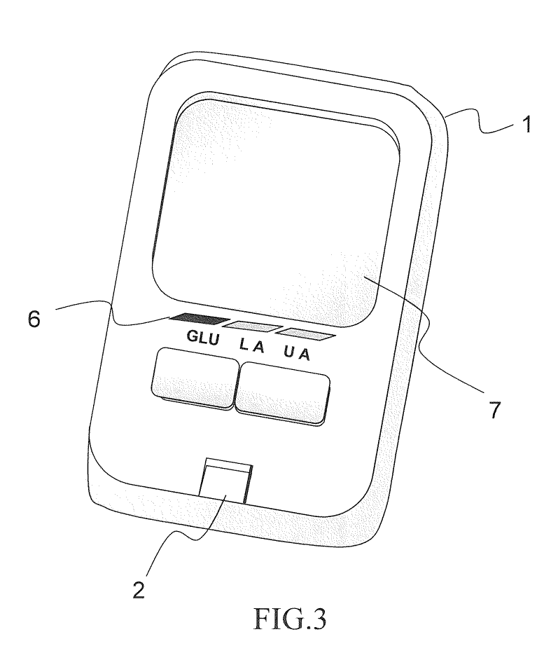

Refer to FIG. 3 a diagram schematically showing the light presented by an indicating unit of a physiological test device according to the present invention. As shown in FIG. 3, the indicating unit 6 is in form of light emitting elements. The indicating unit 6 can use lights to statically or dynamically highlight/present the types or test results of test pieces or memory cards. For examples, the indicating unit 6 may use different colors of lights to indicate the types of test pieces or memory cards; the indicating unit 6 may use lights at different positions of the appearance of the physiological test device 1 to indicate the types or test results of test pieces or memory cards; the indicating unit 6 may use different flashing frequencies of lights in the appearance of the physiological test device 1 to indicate different test results of test pieces or memory cards.

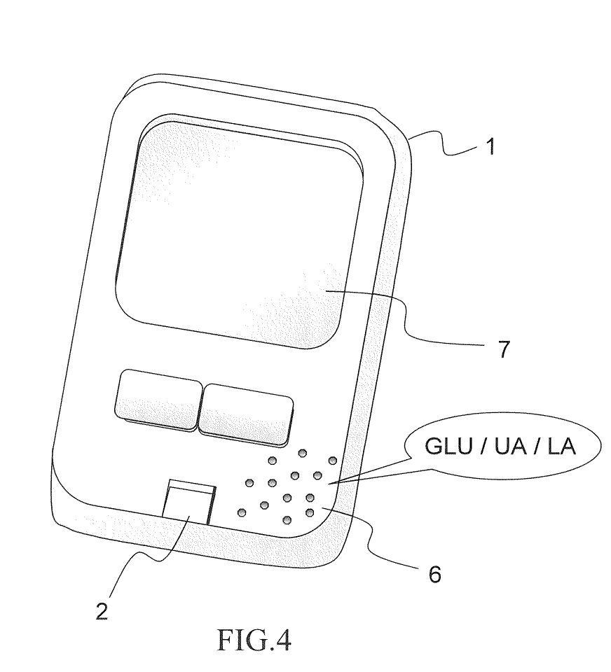

Refer to FIG. 4 a diagram schematically showing that an indicating unit of a physiological test device issues vocal messages according to the present invention. As shown in FIG. 4, the indicating unit 6 is in form of an audio device, such as a speaker, a sound generator or a sound generation circuit, to present audio messages indicating the types or test results of test pieces or memory cards. The audio message may be voice or music indicating the user of his physiological status.

Concluded from the embodiments described above, the present invention is a novel physiological test device, which is used to test multiple physiological parameters in vitro and able to indicate the current test item with lights, sounds, images, text or a combination thereof lest the user be confused by the multiple functions of the test device or different test results thereof. The present invention can prevent the user from mistaking the test type or test result, and thus can overcome the conventional problems that the multiple functions of a single test device confuse the user.

The present invention has the following advantages:
1. The present invention can undertake several types of in-vitro physiological tests. Further, the present invention can indicate the type of the test undertaken currently and present the test result thereof with lights, sounds, images, text or multimedia.
2. The present invention can solve the problem of recognizing the type of test in a multi-functional test device and prevent the user from being confused by the multiple functions of the device. Thus, the user will not take a wrong test piece or obtain a wrong test result.

The embodiments described above are only to exemplify the present invention but not to limit the scope of the present invention. Any equivalent modification or variation according to the spirit of the present invention is to be also included within the scope of the present invention.

What is claimed is:

1. A physiological test device used to test multiple physiological parameters in vitro and comprising
an insertion slot receiving a test piece or a memory card;
a signal sensing unit electrically connected to said insertion slot and receiving a test signal;
an identification unit electrically connected to said insertion slot and identifying a type of a test piece or a memory card inserted into said insertion slot;
a control unit processing identification information provided by said identification unit, performing a test corresponding to said identification information, and performing a computation of said test;
an indicating unit controlled by said control unit to issue an indication signal to present a type or a test result of said test piece or said memory card; and
a display unit using sounds, images or multimedia to statically or dynamically present a test result of said test piece or said memory card.

2. A physiological test device used to test multiple physiological parameters in vitro and comprising
an insertion slot receiving a test piece or a memory card;
a signal sensing unit detecting a signal when said test piece or said memory card is tested;
an identification unit containing information of types of tests implemented by said test pieces or said memory cards to be inserted into said insertion slot;
a control unit receiving and processing a sensation signal from said signal sensing unit to obtain a test result, and receiving an identification result from said identification unit;
an indicating unit controlled by said control unit to issue an indication signal to present a type or a test result of said test piece or said memory card; and
a display unit using sounds, images or multimedia to statically or dynamically present a test result of said test piece or said memory card.

3. The physiological test device according to claim 1, wherein said indication signal is a light, a sound or an image.

4. The physiological test device according to claim 2, wherein said indication signal is a light, a sound or an image.

5. The physiological test device according to claim 1, wherein said test piece has an identification label generating an electric signal, a mechanical signal or an optical signal.

6. The physiological test device according to claim 2, wherein said test piece has an identification label generating an electric signal, a mechanical signal or an optical signal.

7. The physiological test device according to claim 1, wherein said indicating unit uses different colors of lights to indicate different types of tests corresponding to said test pieces or said memory cards.

8. The physiological test device according to claim 2, wherein said indicating unit uses different colors of lights to indicate different types of tests corresponding to said test pieces or said memory cards.

9. The physiological test device according to claim 1, wherein said indicating unit uses lights at different positions on the appearance of said physiological test device to indicate different test results of said test pieces or said memory cards.

10. The physiological test device according to claim 2, wherein said indicating unit uses lights at different positions on the appearance of said physiological test device to indicate different test results of said test pieces or said memory cards.

11. The physiological test device according to claim 1, wherein said indicating unit uses different flashing frequencies of lights on the appearance of said physiological test device to indicate different test results of said test pieces or said memory cards.

12. The physiological test device according to claim 2, wherein said indicating unit uses different flashing frequencies of lights on the appearance of said physiological test device to indicate different test results of said test pieces or said memory cards.

13. The physiological test device according to claim 1, wherein said indicating unit uses different music or audio effects to indicate different test results of said test pieces or said memory cards.

14. The physiological test device according to claim 2, wherein said indicating unit uses different music or audio effects to indicate different test results of said test pieces or said memory cards.

15. The physiological test device according to claim 1, wherein said indicating unit uses different vocal messages to indicate different test results of said test pieces or said memory cards.

16. The physiological test device according to claim 2, wherein said indicating unit uses different vocal messages to indicate different test results of said test pieces or said memory cards.

17. The physiological test device according to claim 1, wherein said display unit is an LCD (Liquid Crystal Display) panel.

18. The physiological test device according to claim 2, wherein said display unit is an LCD (Liquid Crystal Display) panel.

19. The physiological test device according to claim 1, wherein said display unit is an OLED (Organic Light Emitting Diode) panel.

20. The physiological test device according to claim 2, wherein said display unit is an OLED (Organic Light Emitting Diode) panel.
